(19) United States
(12) Patent Application Publication
(10) Pub. No.: US 2010/0108554 A1
(43) Pub. Date: May 6, 2010

(54) Gender-specific, disposable absorbent articles

(76) Inventors: Shannon Kathleen Melius, Appleton, WI (US); Amanda Jean Simon, Oshkosh, WI (US); Marie Elizabeth Luna, Appleton, WI (US); James R. Schermerhorn, Neenah, WI (US); Marcille Faye Ruman, Oshkosh, WI (US); Wayne David Beyer, Appleton, WI (US); Todd William Wilkes, Appleton, WI (US); Katherine C. Wheeler, Menasha, WI (US); Patsy A. Krautkramer, Omro, WI (US)

Correspondence Address: KIMBERLY-CLARK WORLDWIDE, INC.;Tara Pohlkotte, 401 NORTH LAKE STREET, NEENAH, WI, 54956, US

(21) Appl. No.: 12/290,901

(22) Filed: Nov. 4, 2008

(51) Int. Cl. A61B 19/02; B65D 85/00; A61F 13/15

(52) U.S. Cl. 206/438; 206/459.1; 206/459.5; 604/385.28; 604/385.29; 604/385.25

(57) ABSTRACT

Disposable absorbent articles product designs tailored to the differing needs and preferences of males and females are disclosed. In particular embodiments, an array of gender-specific, disposable, absorbent articles comprises a package of first disposable absorbent articles, the package bearing indicia indicating that the articles therein are adapted to be worn by females, and a package of second disposable absorbent articles, the package bearing indicia indicating that the articles therein are adapted to be worn by males. The first disposable absorbent articles differ from the corresponding second disposable absorbent articles in any of a variety of aspects to customize the products for each gender. In certain embodiments, the array includes one product adapted for use by one gender, and a unisex product adapted for use by either gender.

### BACKGROUND

People rely on disposable absorbent products in their everyday lives. Many manufacturers seek to better meet the needs of users of such products. Some manufacturers, for example, have sought to develop products that absorb better. Others have sought to improve fit. Others have sought to improve the visual appeal of the products.

Males and females have different body characteristics, and, as a result, have differing protection needs when utilizing disposable absorbent articles. As an initial matter, males and females of course have different urological physiology which causes urine to exit their respective bodies at different locations relatively to one another. Moreover, men and women have different body compositions (e.g., percentage of body mass consisting of muscle), different skeletal structures, and age differently. As men get older they tend to lose muscle mass in their legs, resulting in thinner legs. In fact, the circumference of the leg of a typical male user of an adult-incontinence article may actually be smaller than the circumference of the leg of a typical female user of an adult-incontinence article—a counterintuitive finding. Also, for the anatomical reasons noted above, men typically release urine at a location further toward the front of the body, while women release urine at a location generally lower on the body compared to men. Furthermore, the shape of a man's hips and groin area is different from a woman's hips and groin area. Men and women may also have varying preferences regarding the feel and appearance of products, such as in relation to material tactile characteristics and product shape.

We are not aware of product designs, product lines, or ways of marketing that address these and other differing body characteristics of males and females who use disposable absorbent articles.

### SUMMARY

We have conceived disposable absorbent product designs tailored to the differing anatomical features and/or tactile and aesthetic preferences of males and females. Furthermore, we have studied and taken into account the differing ways in which males and females age and typically distribute their body weight. Accordingly, we have conceived products, product lines, and ways of drawing attention to these products that address the aforementioned anatomical features, including those correlating with aging.

In one aspect, the present invention is directed to an array of gender-specific, disposable, absorbent articles. In one embodiment, the array comprises a package of first disposable absorbent articles, the package bearing indicia indicating that the articles therein are adapted to be worn by females, each first disposable absorbent article having a first product length; and a package of second disposable absorbent articles, the package bearing indicia indicating that the articles therein are adapted to be worn by males, each second disposable absorbent article having a second product length. The first product length is different from the second product length.

In another embodiment, the array comprises a package of first disposable absorbent articles, the package bearing indicia indicating that the articles therein are adapted to be worn by females, each first disposable absorbent article having a first crotch length; and a package of second disposable absorbent articles, the package bearing indicia indicating that the articles therein are adapted to be worn by males, each second disposable absorbent article having a second crotch length. The first crotch length is different from the second crotch length.

In another embodiment, the array comprises a package of first disposable absorbent articles, the package bearing indicia indicating that the articles therein are adapted to be worn by females, each first disposable absorbent article having an first absorbent core; and a package of second disposable absorbent articles, the package bearing indicia indicating that the articles therein are adapted to be worn by males, each second disposable absorbent article having a second absorbent core. Only one of the first absorbent core and the second absorbent core includes a supplemental absorbent layer, said supplemental absorbent layer comprising a stabilized absorbent.

In another embodiment, the array comprises a package of first disposable absorbent articles, the package bearing indicia indicating that the articles therein are adapted to be worn by females; and a package of second disposable absorbent articles, the package bearing indicia indicating that the articles therein are adapted to be worn by males. Either each first disposable absorbent article has a first graphic visible when viewing an inner, body-facing surface of the first disposable absorbent article, each second disposable absorbent article has a second graphic visible when viewing an inner, body-facing surface of the second disposable absorbent article, or both each first disposable absorbent article has a first graphic visible when viewing the inner, body-facing surface of the first disposable absorbent article and each second disposable absorbent article has a second graphic visible when viewing the inner, body-facing surface of the second disposable absorbent article.

In another embodiment, the array comprises a package of first disposable absorbent articles, the package bearing indicia indicating that the articles therein are adapted to be worn by females; and a package of second disposable absorbent articles, the package bearing indicia indicating that the articles therein are adapted to be worn by males. The package of first disposable absorbent articles has a first fragrance, and the package of second disposable absorbent articles has either (1) a second fragrance different from the first fragrance or (2) no fragrance.

In another embodiment, the array comprises a package of first disposable absorbent articles, the package bearing indicia indicating that the articles therein are adapted to be worn by females, each first disposable absorbent article having a first leg elastic member; and a package of second disposable absorbent articles, the package bearing indicia indicating that the articles therein are adapted to be worn by males, each second disposable absorbent article having a second leg elastic member. The first leg elastic member extends along a first path and the second leg elastic member extends along a second path that has a different shape than the first path.

In another embodiment, the array comprises a package of first disposable absorbent articles, the package bearing indicia indicating that the articles therein are adapted to be worn by females, each first disposable absorbent article having a separately attached first waistband; and a package of second disposable absorbent articles, the package bearing indicia indicating that the articles therein are adapted to be worn by males, each second disposable absorbent article having a separately attached second waistband. The first waistband differs from the second waistband in at least one of width and tension.

In another embodiment, the array comprises a package of first disposable absorbent articles, the package bearing indicia indicating that the articles therein are adapted to be worn by females, each first disposable absorbent article having a first chassis design; and a package of second disposable absorbent articles, the package bearing indicia indicating that the articles therein are adapted to be worn by males, each second disposable absorbent article having a second chassis design. The first chassis design is different than the second chassis design. In a particular version of this embodiment, each first disposable absorbent article comprises a front portion, a back portion, and a crotch portion, wherein the front portion, the back portion, and the crotch portion are integrally connected in the first chassis design, and wherein each second disposable absorbent article comprises a front portion, a back portion, and a crotch portion, wherein the front portion is indirectly and non-integrally connected to the back portion by way of the crotch portion positioned longitudinally intermediate to and bonded to both the front portion and the back portion in the second chassis design.

In another embodiment, the array comprises a package of first disposable absorbent articles, the package bearing indicia indicating that the articles therein are adapted to be worn by females, each first disposable absorbent article having a set of first containment flaps; and a package of second disposable absorbent articles, the package bearing indicia indicating that the articles therein are adapted to be worn by males, each second disposable absorbent article having a set of second containment flaps. The set of first containment flaps differs in at least one structural aspect from the set of second containment flaps.

### DRAWINGS

FIG. 1 depicts one representative example of a disposable adult-incontinence article adapted to be worn by a male in an open, laid-flat orientation.

FIG. 2 depicts one representative example of a disposable adult-incontinence article adapted to be worn by a female in an open, laid-flat orientation.

FIG. 3 depicts one representative example of a disposable adult-incontinence article adapted to be worn by a male in an open, laid-flat orientation.

FIG. 4 depicts one representative example of a disposable adult-incontinence article adapted to be worn by a female in an open, laid-flat orientation.

### DESCRIPTION

Within the context of this specification, each term or phrase below includes the following meaning or meanings:

“Attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Coform” refers to a blend of meltblown fibers and absorbent fibers such as cellulosic fibers that can be formed by air forming a meltblown polymer material while simultaneously blowing air-suspended fibers into the stream of meltblown fibers. The coform material may also include other materials, such as superabsorbent materials. The meltblown fibers and absorbent fibers are collected on a forming surface, such as provided by a foraminous belt. The forming surface may include a gas-pervious material that has been placed onto the forming surface.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

“Disposable” refers to articles which are designed to be discarded after a limited use rather than being laundered or otherwise restored for reuse.

The terms “disposed on,” “disposed along,” “disposed with,” or “disposed toward” and variations thereof are intended to mean that one element can be integral with another element, or that one element can be a separate structure bonded to or placed with or placed near another element.

“Fiber” refers to a continuous or discontinuous member having a high ratio of length to diameter or width. Thus, a fiber may be a filament, a thread, a strand, a yarn, or any other member or combination of these members.

“Layer” when used in the singular can have the dual meaning of a single element or a plurality of elements.

“Liquid impermeable,” when used in describing a layer or multi-layer laminate means that liquid, such as urine, will not pass through the layer or laminate, under ordinary use conditions, in a direction generally perpendicular to the plane of the layer or laminate at the point of liquid contact.

“Liquid permeable” refers to any material that is not liquid impermeable.

“Meltblown” refers to fibers formed by extruding a molten thermoplastic material through a plurality of fine, usually circular, die capillaries as molten threads or filaments into converging high velocity gas (e.g., air) streams, generally heated, which attenuate the filaments of molten thermoplastic material to reduce their diameters. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblowing processes can be used to make fibers of various dimensions, including macrofibers (with average diameters from about 40 to about 100 microns), textile-type fibers (with average diameters between about 10 and 40 microns), and microfibers (with average diameters less than about 10 microns). Meltblowing processes are particularly suited to making microfibers, including ultra-fine microfibers (with an average diameter of about 3 microns or less). A description of an exemplary process of making ultra-fine microfibers may be found in, for example, U.S. Pat. No. 5,213,881 to Timmons, et al. Meltblown fibers may be continuous or discontinuous and are generally self bonding when deposited onto a collecting surface.

“Member” when used in the singular can have the dual meaning of a single element or a plurality of elements.

“Nonwoven” and “nonwoven web” refer to materials and webs of material that are formed without the aid of a textile weaving or knitting process. For example, nonwoven materials, fabrics or webs have been formed from many processes such as, for example, meltblowing processes, spunbonding processes, air laying processes, and bonded carded web processes.

These terms may be defined with additional language elsewhere in the specification.

It should be noted that a number of the features in this invention implicate a measurement of length. In many cases a simple rule may be used to measure the distance from one edge to another. Of course, more sophisticated techniques may be used (e.g., when measuring the perimeter of the leg opening, which can comprise a series of curves). For example, photomicrographs can be prepared and analyzed. Such images could be digitized and appropriate algorithms used to measure length. Alternatively, mechanical devices may be used. Because many of the inventive versions are directed to arrays comprising two products, it is important that the same measurement technique be used when comparing features (e.g., anterior length) of two products in an array. References to length and width dimensions are with respect to the product positioned in an open, laid-flat configuration.

Various embodiments of the invention shall now be discussed. Many of the various aspects of the present invention which focus on the gender-specific product solutions directed to the unique needs of the different genders are applicable to infant, child, and adult alike. However, many of the illustrative examples will be presented in the context of gender-specific adult incontinence articles.

Generally, the gender-specific, disposable, adult-incontinence articles employed in certain aspects of the present invention are made using methods and processes known in the art, but configured to produce one or more features in the article adapted to be worn by females that are different than one or more features in the article adapted to be worn by males. As noted above, analysis of the physiological characteristics of males and females resulted in gender-specific designs, the features of which are described in more detail in the following paragraphs.

One representative version of the invention is an array of disposable adult-incontinence articles. The array can include a first disposable adult-incontinence article having a first anterior length, wherein the first article is adapted to be worn by women; and a second disposable adult-incontinence article having a second anterior length, wherein the second article is adapted to be worn by men, and wherein the first anterior length is less than the second anterior length. For purposes of this application, the term “anterior length” refers to the shortest distance between the waist-opening front edge and the leg-opening front edge.

Another representative version of the invention is an array of disposable incontinence articles. The array can include a first disposable adult-incontinence article having a first leg-opening front edge, wherein the first article is adapted to be worn by women; and a second disposable adult-incontinence article having a second leg-opening front edge, wherein the second article is adapted to be worn by men, and wherein the first leg-opening front edge is more convex toward the waist-opening front edge than the second leg-opening front edge. That is, the boundary defined by the first leg-opening front edge of the article adapted to be worn by women curves more toward the waist-opening front edge (and away from the open space bounded or partially bounded by the leg-opening edges) compared to the boundary defined by the second leg-opening front edge of the article adapted to be worn by men. For purposes of this application, a women's article having this feature comprises a leg-opening front edge having (or, alternatively, establishing a boundary having) a greater convexity compared to the leg-opening front edge of the corresponding men's article.

Another representative version of the invention is an array of disposable incontinence articles. The array can include a first disposable adult-incontinence article having a first leg-opening back edge, wherein the first article is adapted to be worn by women; and a second disposable adult-incontinence article having a second leg-opening back edge, wherein the second article is adapted to be worn by men, and wherein the first leg-opening back edge is more concave toward the waist-opening back edge than the second leg-opening back edge. That is, the boundary defined by the first leg-opening back edge of the article adapted to be worn by women curves more away from the waist-opening back edge (and toward the open space bounded or partially bounded by the leg-opening edges) compared to the boundary defined by the second leg-opening back edge of the article adapted to be worn by men. For purposes of this application, a women's article having this feature comprises a leg-opening back edge having (or, alternatively, establishing a boundary having) a greater concavity compared to the leg-opening back edge of the corresponding men's article.

Another representative version of the invention is an array of disposable adult-incontinence articles. The array can include a first disposable adult-incontinence article having a first crotch width, wherein the first article is adapted to be worn by women; and a second disposable adult-incontinence article having a second crotch width, wherein the second article is adapted to be worn by men; and wherein the first crotch width is less than the second crotch width. For purposes of this application, the term “crotch width” refers to the shortest distance between the outside boundaries of the opposing, elastic members closest to the opposing leg-opening side edges. For example, if three elastic strands are employed near each leg-opening side edge, then “crotch width” equates to the shortest distance between: (1) of the three elastic strands proximate to a first leg-opening side edge, the outside boundary of that strand closest to the first leg-opening side edge; and (2) of the three elastic strands proximate to the opposing, second leg-opening side edge, the outside boundary of that strand closest to the opposing, second leg-opening side edge.

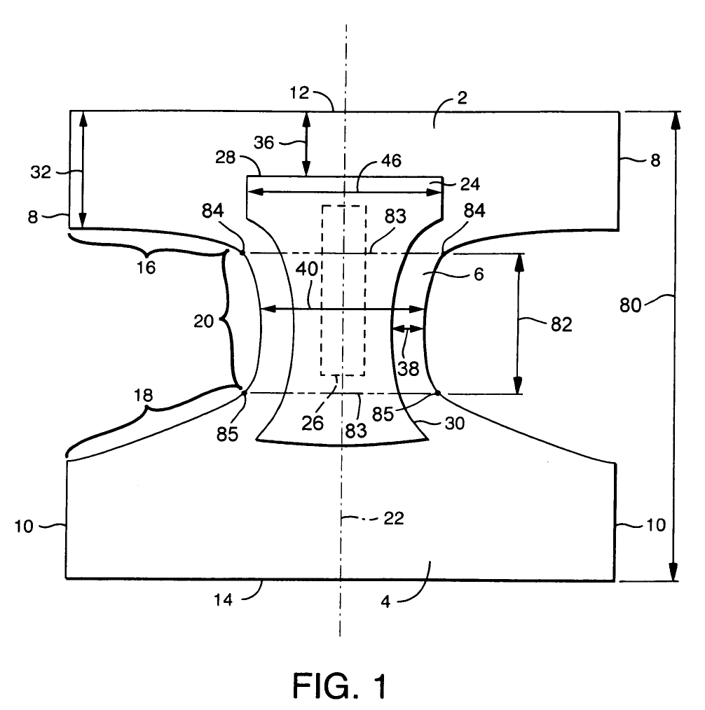

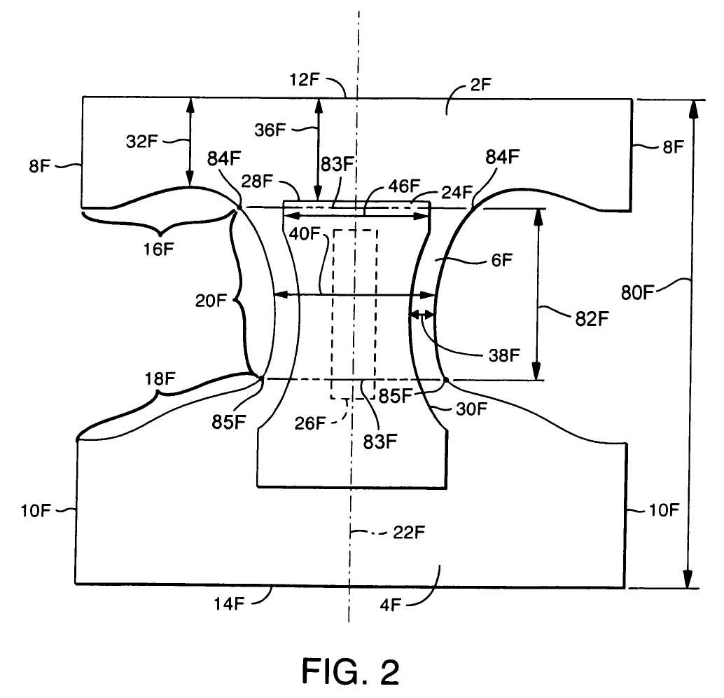

Another representative version of the invention is an array of disposable adult-incontinence articles. The array can include a first disposable adult-incontinence article having a first gasket width, wherein the first article is adapted to be worn by women; and a second disposable adult-incontinence article having a second gasket width, wherein the second article is adapted to be worn by men; and wherein the first gasket width is less than the second gasket width. For purposes of this application, the term “gasket width” refers to, in the crotch portion, the distance, along a line bisecting the middle of the leg-opening side edges in a transverse direction parallel to the waist-opening edges (see FIGS. 1 and 2 and accompanying text), between a side edge of the absorbent core (e.g., a fluff/superabsorbent core) and the outside boundary of the elastic member closest to the nearest, leg-opening side edge If, as is likely, the absorbent core, such as a core comprising fluff and superabsorbent, is disposed between a liquid-permeable, body-facing liner and a liquid-impermeable backsheet (as with an absorbent insert or assembly comprising an absorbent core sandwiched between a liner and backsheet), then the side edge of the absorbent core is located at the boundary of the fluff/superabsorbent core, not at the boundary of any substrate that helps encase and extends beyond the absorbent core.

Another representative version of the invention is an array of disposable adult-incontinence articles. The array can include a first disposable adult-incontinence article having a first leg-opening perimeter, wherein the first article is adapted to be worn by women; and a second disposable adult-incontinence article having a second leg-opening perimeter, wherein the second article is adapted to be worn by men; and wherein the first leg-opening perimeter is greater than the second leg-opening perimeter. For purposes of this application, the term “leg-opening perimeter” refers to the perimeter of a leg opening in a substantially flat, laid-open, disposable adult-incontinence article. The exemplary articles displayed in FIGS. 1 and 2 are in a substantially flat, laid-open condition.

As noted generally above, the present invention involves various combinations of the aforementioned characteristics including one, some, or all of the aforementioned characteristics.

Another representative version of the invention is an array of disposable adult-incontinence articles. The array can include a first disposable adult-incontinence article having a first graphic disposed thereon, wherein the first article is adapted to be worn by women; and a second disposable adult-incontinence article having a second graphic disposed thereon, wherein the second article is adapted to be worn by men; and wherein the first graphic is different than the second graphic. In one exemplary embodiment, the first and second graphic comprise one or more stripes disposed on material proximate to the waist portion of the articles, but of a different color (e.g., one or more blue stripes on the article adapted to be worn by men; and one or more pink stripes on the article adapted to be worn by women). Of course such graphics may be employed in combination with one or more of the inventive features discussed above.

Another representative version of the invention is an array of disposable adult-incontinence articles comprising one or more of the gender-specific features discussed above or in the following paragraphs, with the first disposable adult-incontinence article further comprising an absorbent having a first waist/absorbent distance, wherein the first article is adapted to be worn by women; and a second disposable adult-incontinence article having a second waist/absorbent distance, wherein the second article is adapted to be worn by men; and wherein the first waist/absorbent distance is more than the second waist/absorbent distance. The term “waist/absorbent distance” refers to the minimum distance between the front edge of the waist opening and front edge of the absorbent core. Again, as discussed above, the edge of the absorbent core refers to, for example, the edge of a fluff/superabsorbent core (not the edge of substrates that help contain the core if, as may be the case, the edge of these substrates extend beyond the boundary of the absorbent core itself.

Another representative version of the invention is an array of disposable adult-incontinence articles comprising one or more of the dimensional (e.g., those inventive features directed to a length, width, distance, or other such measurement) or ornamental (e.g., those inventive features directed to disposing a graphic on an article) gender-specific features discussed above or in the following paragraphs, with the first disposable adult-incontinence article further comprising an absorbent core having a first frontal area, wherein the first article is adapted to be worn by women; and a second disposable adult-incontinence article having an absorbent core having a second frontal area, wherein the second article is adapted to be worn by men; and wherein the first frontal area is less than the second frontal area. The term “frontal area,” when describing the absorbent core (e.g., a fluff/superabsorbent core), refers to the area of the body-facing surface of the absorbent core forward of an imaginary transverse line bisecting the article, with area determined when the article comprising the absorbent core is in a substantially flat, laid-open position (e.g., for some articles, the substantially flat, laid-open position generally corresponds to an hourglass-like shape). For example, if an article is in a substantially flat, laid-open position, and has an hourglass-like shape like those versions depicted in FIGS. 1 and 2 (and which have a shape generally analogous to the capital letter “I”), an imaginary transverse line would be a horizontal line halfway between the waist-opening front edge and the waist-opening back edge (i.e., the imaginary transverse line would be a horizontal line half-way between the upper and lower horizontal lines that make up the letter “I”).

Another representative version of the invention is an array of disposable adult-incontinence articles comprising one or more of the dimensional (e.g., those inventive features directed to a length, width, distance, or other such measurement) or ornamental (e.g., those inventive features directed to disposing a graphic on an article) gender-specific features discussed above or in the following paragraphs, with the first disposable adult-incontinence article further comprising an absorbent core having a first frontal transverse span, wherein the first article is adapted to be worn by women; and a second disposable adult-incontinence article having an absorbent core having a second frontal transverse span, wherein the second article is adapted to be worn by men; and wherein the first frontal transverse span is less than the second frontal transverse span. The term “frontal transverse span,” when describing the absorbent core (e.g., a fluff/superabsorbent core), refers to the longest distance, in a transverse direction parallel to the waist-opening edges (see FIGS. 1 and 2 and accompanying text), between the opposing, longitudinal edges of the absorbent core forward of an imaginary transverse line bisecting the article in a substantially flat, laid-open position (e.g., for many articles, the substantially flat, laid-open position generally corresponds to a hourglass-like shape).

Another representative version of the invention is an array of disposable adult-incontinence articles comprising one or more of the inventive dimensional (e.g., those inventive features directed to a length, width, distance, or other such measurement), ornamental (e.g., those inventive features directed to disposing a graphic on an article) or other gender-specific features discussed above or in the following paragraphs, with the first disposable adult-incontinence article contained in a first package having a first statement disposed thereon, wherein the first article is adapted to be worn by women; and a second disposable adult-incontinence article contained in a second package having a second statement disposed thereon, wherein the second article is adapted to be worn by men; wherein the first statement associates the article with use by women and the second statement associates the article with use by men. The statement could, for example, take the form of indicia, such as printed indicia, including printed text or figures.

Another representative version of the invention is a method of providing a gender-specific, disposable, adult-incontinence article, the method comprising the steps of transmitting a statement embodied in a tangible medium that refers to an array of gender-specific, disposable, adult-incontinence articles; and positioning in the hands of a user a disposable, adult-incontinence article adapted to fit the gender of the user.

In other representative versions of the invention, a gender-specific array of disposable, adult-incontinence articles comprises a gender-specific, disposable, adult-incontinence article comprising one or more of the inventive dimensional (e.g., those inventive features directed to a length, width, distance, or other such measurement), ornamental (e.g., those inventive features directed to disposing a graphic on an article) or other gender-specific features discussed above or in the following paragraphs, and a unisex disposable, adult-incontinence article. In other words, rather than a comparison being made between an article adapted to be worn by males (such as men) and an article adapted to be worn by females (such as women), the comparison is made between either: (1) an article adapted to be worn by women and a unisex article; or (2) an article adapted to be worn by men and a unisex article. “Unisex” refers to an article that can be worn by either a man or a woman. While the comparison is being made between a unisex article and either an article adapted to be worn by a man or an article adapted to be worn by a women, the relationships described above still hold, with the exception being that a unisex article is substituted for that article not present in the array. In other words, if the array was made up of a disposable, adult-incontinence article adapted to be worn by women, and a unisex article (and therefore an article adapted to be worn by men is absent from the array), then, for example, the women's article may comprise: (1) a leg-opening front edge defining a boundary that is more convex in a direction toward the waist-opening front edge compared to the boundary for a unisex article; (2) a leg-opening back edge defining a boundary that is more concave in a direction toward the waist-opening back edge compared to the boundary for a unisex article; and so on. If, on the other hand, the array is made up of a disposable, adult-incontinence article adapted to be worn by men, and a unisex article (and therefore an article adapted to be worn by women is absent from the array), then, for example, the man's article might have: (1) a gasket width greater than the gasket width for the unisex article; (2) a waist/absorbent distance less than the waist/absorbent distance of the unisex article; and so on.

For purposes of this application, “fit features” refer to one or more of anterior length, shape of leg-opening front edge (i.e., degree of convexity of said edge), shape of leg-opening back edge (i.e., degree of concavity of said edge), leg-opening perimeter, and gasket width. Also, for purposes of this application, “leg-opening features” refers to one or more of anterior length, shape of leg-opening front edge, shape of leg-opening back edge, and leg-opening perimeter.

Other versions of the invention can include a disposable, adult-incontinence article comprising a fit feature adapted for a specific gender. Still other versions of the invention can comprise various combinations of those features denominated as fit features, and which are adapted for a specific gender.

Other versions of the invention can include a disposable, adult-incontinence article comprising a leg-opening feature adapted for a specific gender. Other versions of the invention can comprise various combinations of those features denominated as leg-opening features, and which are adapted for a specific gender.

The various representative embodiments described in the preceding two paragraphs may, of course, be contained in packages on which are disposed statements associating the articles contained therein with use by a specific gender. Furthermore, another representative version of the invention is a method of providing a gender-specific, disposable absorbent article, the method comprising the steps of transmitting a statement embodied in a tangible medium that refers to a gender-specific, disposable absorbent article comprising a fit feature and/or a leg-opening feature; and positioning in the hands of a user a disposable, adult-incontinence article comprising a fit feature and/or a leg-opening feature, wherein the article adapted to fit the gender of the user.

FIG. 1 depicts an exemplary version of a disposable, adult-incontinence article adapted to be worn by a man. The figure gives a top plan view of a substantially flat, laid-open article before attachment of the side edges of the front portion to the side edges of the back portion to form an article having a waist opening and two leg openings. The front portion, back portion, and their respective side edges are identified below.

As noted above, the article includes a front portion 2 and a back portion 4. The front portion and back portion are interconnected by a crotch portion 6. Generally the term “chassis” is used to refer to the combination comprising a front portion, a back portion, and a crotch portion connecting said front portion to said back portion. The front portion has two side edges 8 and the back portion has two side edges 10. When assembling the article, the side edges 8 are attached to the side edges 10 to form a shape similar to that of underwear and having a waist opening and two leg openings. If desired, the side edges may be releasably engaged to one another (e.g., by using mechanical fasteners such as hook-and-loop-type materials; adhesives; etc.). Also, the side edges of the front and back portions need not be attached directly to one another, but may be attached to one or more intermediate elements (e.g., side panels—not shown in the figures).

The front portion also has a waist-opening front edge 12, and the back portion has a waist-opening back edge 14. When the side edges are joined to one another the waist-opening edges are connected to form a perimeter that typically fits around the lower torso of a user.

For purposes of this application, the edges defining the leg opening are categorized as including a leg-opening front edge 16, a leg-opening back edge 18, and a leg-opening side edge 20. Typically a major portion of the leg-opening side edge runs somewhat parallel to an imaginary longitudinal centerline 22 running from the back to the front of the article. Major portions of the leg-opening front and back edges will typically not run parallel to the longitudinal center line. The leg-opening is categorized in this way to help distinguish certain features of gender-specific articles from one another, as discussed below.

The article will typically include an absorbent core 24. In the depicted representative example, the absorbent core includes fluff (not shown), super absorbent material (not shown), and an optional surge material 26 adapted to help rapidly take in and distribute urine for absorption by the fluff/superabsorbent material. The absorbent core includes an absorbent front edge 28, and absorbent side edges 30. For those articles made by attaching an absorbent assembly or absorbent insert to, for example, an hourglass-like substrate, with the assembly comprising an absorbent core sandwiched between a liquid-permeable, body-side liner, and a liquid-impermeable backing, the liner, backing, or both may extend beyond the edge of the absorbent core itself. For example, in some articles, the liner and backing (i.e., the backsheet of the absorbent core insert) are attached to one another around the perimeter of the absorbent core contained therein, and the area of attachment of these materials may form a flexible flange extending beyond the edge of the absorbent core. Such a flange is not depicted in FIG. 1 or FIG. 2.

These various edges help further define one or more distances that can vary between a disposable, adult-incontinence article adapted to be worn by a man, and a disposable, adult-incontinence article adapted to be worn by a woman. As noted earlier, the anterior length 32 is the shortest distance between the leg-opening front edge 16 and the waist-opening front edge 12. It should be noted that for purposes of clearly depicting the anterior length in FIG. 1, the two-headed arrow representing this distance is slightly offset from one of the side edges 8 of the front portion. If, however, this side edge corresponded to the shortest distance between the leg-opening front edge 16 and the waist-opening front edge 12, then the anterior length 32 would actually overlay the side edge 8.

The product length 80 is the shortest distance between the waist-opening front edge 12 and the waist-opening back edge 14.

The waist/absorbent distance 36 is the shortest distance between the absorbent core front edge 28 and the waist-opening front edge 12.

The gasket width 38 is the distance, in the crotch portion, along a line bisecting the middle of the leg-opening side edges and in a transverse direction, between the absorbent core side edge 30 and the outer boundary of the elastic member closest to leg-opening side edge 20. It should be noted that in FIG. 1, the two-headed arrow representing this distance touches the leg-opening side edge 20 because of the difficulty in representing the small distances typically characterizing the length between the outer boundary of the elastic member closest to the side edge, and the side edge itself. Of course, in reality, if the outer boundary of the elastic member closest to the leg-opening side edge is inward of the side edge, as is likely the case, then gasket width is measured to the outer boundary of the elastic member—not to the side edge itself.

The crotch width 40 is defined as the shortest distance between the outside boundaries of the opposing, elastic members closest to the opposing leg-opening side edges 20. It should be noted that in FIG. 1, the two-headed arrow representing this distance touches the leg-opening side edges 20 because of the difficulty in representing the small distances typically characterizing the length between the outer boundary of the elastic member closest to the side edge, and the side edge itself.

The crotch length 82 is defined as the shortest longitudinal distance between two straight, transversely extending imaginary lines 83, one of which passes through both of the front endpoints 84 of the leg-opening side edges 20, and the other of which passes through both of the back endpoints 85 of the leg-opening side edges 20. The front endpoints 84 of the leg-opening side edges 20 are those points at which the leg-opening side edges 20 extend primarily in a transverse direction, as opposed to primarily in a longitudinal direction. If the leg-opening sides edges 20 are shaped such they gradually curve into the leg-opening front edges 16, then the front endpoints 84 of the leg-opening side edges 20 are those points approaching the leg-opening front edges 16 at which the leg-opening side edges 20 extend at a 45-degree angle to the longitudinal axis of the product. Likewise, the back endpoints 85 of the leg-opening side edges 20 are those points at which the leg-opening side edges 20 extend primarily in a transverse direction, as opposed to primarily in a longitudinal direction. If the leg-opening sides edges 20 are shaped such they gradually curve into the leg-opening back edges 18, then the back endpoints 84 of the leg-opening side edges 20 are those points approaching the leg-opening back edges 18 at which the leg-opening side edges 20 extend at a 45-degree angle to the longitudinal axis of the product.

The frontal area of the absorbent core 24 corresponds to the body-facing surface area of that region of the absorbent core between an imaginary transverse line approximately bisecting the article when in a substantially flat, laid-open position (this imaginary line is not shown in FIGS. 1 and 2, but would approximately overlay the double-headed arrow designated as “40”) and the waist-opening front edge.

The frontal transverse span or distance 46 corresponds to the longest distance, in a transverse direction, between the opposing, longitudinal edges 30 of the absorbent core, for that portion of the absorbent core forward of a location between an imaginary transverse line bisecting the article in a substantially flat, laid-open position and the waist-opening front edge (the location of which, for the depicted, representative embodiment, is discussed in the previous paragraph). It should also be noted that, for the depicted embodiment, the side edges 30 of the absorbent core establish a somewhat complex boundary, with a curvilinear portion in the crotch region, and a linear portion in the front region, with each linear portion extending in a substantially perpendicular fashion toward the front edge of the absorbent core.

FIG. 2 depicts these same features, but for one representative example of a disposable, adult-incontinence article adapted for use by women. The same numbers used in FIG. 1 are used in FIG. 2, with the exception that the numbers in FIG. 2 are appended with the letter “F” to signify that these features correspond to those employed in an article adapted to be worn by women.

Table 1 below gives a comparison of certain features of two articles in an exemplary gender-specific array of articles. As noted above in the Summary section, it is not necessary that gender-specific articles in accordance with the present invention incorporate all or any of the features identified in Table 1. Nor does the invention encompass only those features listed in this table.

TABLE 1

| Feature | Article for Women | Article for Men |
|---|---|---|
| Anterior Length | Shorter | Longer |
| Shape of Leg-Opening | More convex toward | Less convex toward |
| Front Edge | waist-opening front | waist-opening front |
| | edge | edge |
| Shape of Leg-Opening | More concave | Less concave |
| Back Edge | toward waist- | toward waist- |
| | opening back edge | opening back edge |
| Leg-Opening Perimeter | More | Less |
| Crotch Width | Narrower (Shorter) | Wider (Longer) |
| Waist/Absorbent Distance | Longer | Shorter |
| Frontal Area of Absorbent | Less | More |
| Core | | |
| Frontal Transverse Span | Shorter | Longer |
| Graphic Disposed on | Different | Different |
| Article | | |
| Gasket Width | Narrower (Shorter) | Wider (Longer) |

As noted elsewhere, one feature that may be employed in an array of gender-specific articles of the present invention—comprising a disposable, adult-incontinence article adapted to be worn by a woman, and a disposable, adult-incontinence article adapted to be worn by a man—is that the article adapted to be worn by women has a shorter anterior length than the article adapted to be worn by men. This may be achieved, for example, when the leg-opening front edge of the women's article defines a boundary that is more convex toward the waist-opening front edge compared to the boundary defined by the leg-opening front edge of the man's article. It is believed that such convexity helps define a leg opening better suited to the shape of an average female user's thigh compared to the shape of an average male user's thigh. As noted elsewhere, as men age their legs tend to lose muscle mass, which results in thinner legs and a smaller circumference around the upper thigh. Surprisingly, the circumference of the legs of many male users of adult-incontinence products may actually be smaller than the circumference of the legs of many female users of adult-incontinence products. Also, a leg-opening front edge having a shorter anterior length is more likely to result in a boundary that better conforms to the curvature of the upper thigh of the typical female user of the article.

Another feature that may be employed in an array of gender-specific articles is a disposable, adult-incontinence article adapted to be worn by women that comprises a leg-opening back edge that defines a boundary that is more concave in a direction toward the waist-opening back edge compared to the boundary defined by the leg-opening back edge of a corresponding article adapted to be worn by men. A more concave boundary in the woman's article helps ensure coverage of the typical female user's buttocks.

Yet another characteristic that may be selected for use in an array of gender-specific articles is a disposable, adult-incontinence article adapted to be worn by women having a leg-opening perimeter that is greater than the leg-opening perimeter for the corresponding article adapted to be worn by men.

Because of differences in their respective physiologies, an array of gender-specific articles may include a disposable, adult-incontinence article adapted to be worn by women that has a smaller crotch width compared to the corresponding article for men.

Still another feature that may be employed in an array of gender-specific articles is a disposable absorbent article adapted to be worn by a female that has a different product length than the product length for the corresponding article adapted to be worn by a male. Physical characteristics of the target user group as well as style preferences may dictate which product in the array would have the longer length. For example, for an adult incontinence garment, articles adapted for men may, in particular embodiments, desirably have a shorter product length 80 than the product length 80F for the corresponding articles adapted for women, to allow the product to better fit below the bellies of aging men which frequently protrude from the rest of the body. In another example, such as for training or enuresis garments for children, articles adapted for girls may, in particular embodiments, desirably have a shorter product length 80F than the product length 80 for the corresponding articles adapted for boys, to better provide a more discreet, feminine bikini-like fit.

Another feature that may be employed in an array of gender-specific articles is a disposable absorbent article adapted to be worn by a female that has a different crotch length than the crotch length for the corresponding article worn by a male. Physical characteristics of the target user group as well as style preferences may dictate which product in the array would have the longer crotch length. For example, for an adult incontinence garment, articles adapted for men may, in particular embodiments, desirably have a shorter crotch length 82 than the crotch length 82F for the corresponding articles adapted for women, to allow the product to better fit the relatively smaller legs which have been found to be common with aging men. In another example, such as for training or enuresis garments for children, articles adapted for boys may, in particular embodiments, desirably have a longer crotch length 82 than the crotch length 82F for the corresponding articles adapted for girls, to accommodate the physiological characteristics of boys as compared to girls.

The point at which urine exiting the body contacts a disposable absorbent article is different for men and women, given their physiological differences. Accordingly, the position of the absorbent in an article adapted to be worn by men may be shifted forward in the article, resulting in the distance between the waist-opening front edge and the front edge of the absorbent decreasing. This helps ensure that the point at which urine first contacts the article is on the absorbent core. A women's article will tend to employ the absorbent core at a position ensuring contact with a urination point that is not as far forward as that for a man.

Another feature of the present invention relates to the use of a supplemental absorbent layer in either a disposable absorbent article adapted to be worn by a female or the corresponding article adapted to be worn by a male. By providing a supplemental absorbent layer (that is, a second absorbent layer that is stacked on top of the base absorbent layer) positioned to be in the fluid insult target zone for a particular gender, absorbent articles targeted for use by that gender can perform better. For example, in an array of gender-specific absorbent articles, a disposable absorbent article adapted to be worn by females may have a supplemental absorbent layer, such as a separately provided absorbent pledget laid atop the base absorbent (such as in the crotch area), while the corresponding disposable absorbent article adapted to be worn by males has no supplemental absorbent layer. In a contrasting example of an array of gender-specific absorbent articles, a disposable absorbent article adapted to be worn by males may have a supplemental absorbent layer, such as a separately provided absorbent pledget laid atop the base absorbent (such as in the front area), while the corresponding disposable absorbent article adapted to be worn by females has no supplemental absorbent layer. In particular embodiments, the supplemental absorbent layer comprises a stabilized absorbent pledget, such as that disclosed in U.S. Patent Application Publication US 2003/0225384, the entirely of which is incorporated herein by reference to the extent consistent herewith. As used herein, “stabilized absorbent” refers to an absorbent structure that has a quantity of binder material added to a mixture of other absorbent materials, such as a quantity of absorbent fibers and a quantity of superabsorbent material, to provide an absorbent matrix. It should be noted that the absorbent mixture (for example, the quantity of absorbent fibers and quantity of superabsorbent material) and the quantity of binder material may be provided in a homogeneous mixture; or the quantity of binder material may be added to the absorbent mixture in a stratified configuration. The binder material is then activated to bond the resultant absorbent matrix together. Stabilized absorbent structures are advantageously produced “off-line,” by a process separate from the production of the entire absorbent article. Another example of a supplemental absorbent layer is a layer of coform material, defined above.

The frontal area and/or frontal transverse span of the absorbent core may also be varied between an article adapted to be worn by men and an article adapted to be worn by women. Because a man's point of urination may move, the overall frontal area of the absorbent core in a disposable, adult-incontinence article adapted to be worn by men may be greater than the corresponding frontal area in a woman's article. Similarly, the frontal transverse span of the absorbent core in a disposable, adult-incontinence article adapted to be worn by men may be greater than the corresponding frontal transverse span of the absorbent core in a woman's article.

As discussed elsewhere, graphics may be disposed on an article adapted to be worn by women that are different than those disposed on an article adapted to be worn by men. In one example, one or more stripes of a desired width are disposed around the perimeter of the article in a location proximate to the waist-opening front edge, back edge, or both. The shape, color, number, or other characteristic of the stripes disposed on a man's article may be different from those disposed on the woman's article. For example, blue-colored stripes may be disposed around the perimeter of the waist region (i.e., that portion of the article proximate to the waist opening) of a man's article, while pink-colored stripes are disposed around the perimeter of the waist region of the corresponding woman's article. In another representative example, graphics may be disposed on the article so that the article more closely resembles the shape and look of woven underwear. So, for example, a graphic fly or opening may be employed on a man's article so that the article resembles men's woven underwear briefs. In particular embodiments, graphics or images are provided in the article such that they are visible from the inner body-facing surface of the article. For example, such graphics could be printed on the body-facing surface of a body-side layer, on the garment-facing surface of a body-side layer, or on either surface of a material that underlies a body-side layer, so long as the graphic or image is visible when viewing the body-side surface of the finished product. Desirably, such “internal” graphics or images are different between the male and female-adapted gender-specific articles. Alternatively, internal graphics could be present on one gender-specific article, but not on the other, corresponding gender-specific article. For example, a printed faux fly opening could be visible on the inner surface of a male article, while no visible image would be present on the inner surface of the corresponding female article. In additional embodiments, an internally-visible surface of the gender specific articles could be pressure or heat embossed with a design pattern, and the embossed design could be different between the male and female-adapted gender-specific articles. Alternatively, internal embossing could be present on one gender-specific article, but not on the other, corresponding gender-specific article. For example, an embossed faux fly opening could be printed on the inner surface of a male article, while no embossing would be present on the inner surface of the corresponding female article. The various illustrative embodiments of printed graphics and images and embossed images can be used alone or in various combination with each other.

Also, the distance between the edge of the absorbent core and the outermost elastic strand proximate to the nearest leg-opening side edge on a man's disposable adult-incontinence article may be greater than that in the corresponding article for a woman. Due to physiological differences between a man and a woman in the area of the crotch, this additional distance generally corresponds to there being additional substrate between the edge of the absorbent core and the outermost elastic member proximate to the side edge of the nearest leg opening. This substrate, which will typically be flexible and incorporate an elastic member proximate to the leg-opening side edge, acts, in a sense, as a gasket to help contain urine. As mentioned elsewhere, the circumference of an older man's leg tends to be smaller due to the loss of muscle mass, and often may be less than that of a woman. Accordingly, an increased gasket width helps to ensure the presence of sufficient material between the inner surface of a man's thigh and the absorbent core.

The preceding paragraphs outline various features that may be employed in an array of gender-specific, disposable, adult-incontinence articles, with these features differing in some way between an article adapted to be worn by a female and an article adapted to be worn by a male. The article itself may employ many different components and subassemblies, so long as the array of gender-specific articles employs one or more of the novel features discussed elsewhere in this application. For example, the article may comprise an hour-glass-shaped liquid-impermeable substrate, such as a film, attached to an hour-glass-shaped liquid-permeable substrate, such as a nonwoven, with a cellulosic-fluff/superabsorbent-material absorbent core disposed between the liquid-impermeable and liquid-permeable substrate. Either of these substrates can be more complicated of course (e.g., laminates of nonwovens, films, or both; co-form materials; etc.), as can the absorbent core. Also, various pigments, odor-control agents, fillers, and other materials may be employed.

In various embodiments of the present invention, fragrances could be added to the products, the packaging, or both. Preferably, such fragrances would be gender specific. For example, the male product (or packaging for the male product) could be impregnated, coated, or otherwise affected with a fragrance desirable to males, and the female product (or packaging for the female product) could be impregnated, coated, or otherwise affected with a fragrance desirable to females. Alternatively, one gender-specific product could contain a fragrance and the other gender-specific product would contain no fragrance. For example, the female product might contain fragrance and the male product would not. As used herein, when reference is made to a “package” of articles having a fragrance, it is contemplated that the fragrance could be present on the products themselves, the packaging material, or both.

Elastic materials such as strands or webs may be employed to help give the article elastomeric qualities in desired locations. For example, elastic members such as strands are often employed in the vicinity of the leg openings to help ensure a close fit, frequently referred to as “leg elastics.” Such leg elastics could be straight or curved, both configurations being known in the art. New to the art, however, would be the possibility of including gender-specific leg elastic constructions, whereby the shape and dimension of the leg elastic for one gender differs from the shape and dimension of the leg elastic for the other, corresponding gender in the array. Such gender-specificity can be advantageous in customizing the leakage and fit aspects of gender-specific articles. In one example, the leg elastic is curved for one gender, but straight for the other gender. Also, elastic members such as strands are often positioned below the waist-opening to effect a close fit around the lower torso of a user. For example, spaced-apart, substantially parallel elastic strands may be sandwiched between two nonwoven materials in the vicinity of the torso. Often the strands are attached to nonwoven facings, typically using adhesive, when the strands are in a stretched condition. The resulting laminate—elastic strands sandwiched between two nonwoven facings—is then allowed to retract, producing a gathered substrate capable of stretching and recovering its shape.

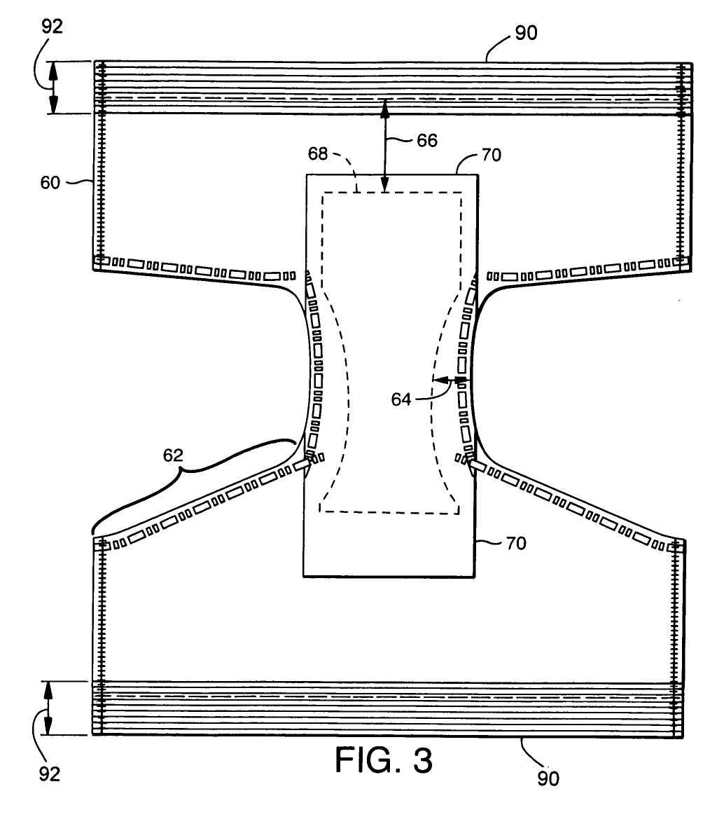

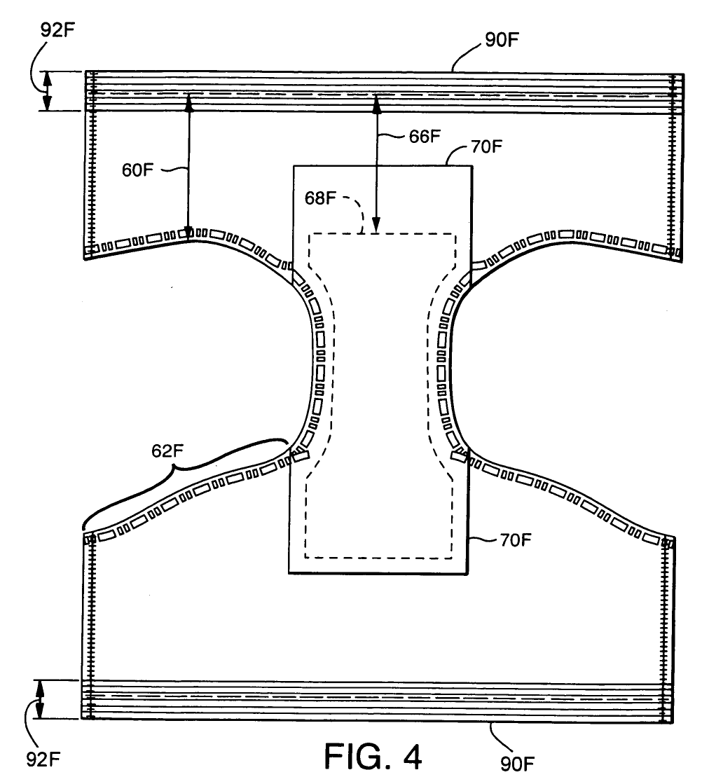

In particular embodiments, one or both of the gender-specific absorbent articles in the array have a separately attached elastomeric waistband 90, as representatively illustrated in FIGS. 3 and 4. Each waistband 90 has a width dimension 92 that extends parallel to the longitudinal direction of the article—that is, from a directional standpoint, the width dimension 92 of the waistband extends from at or near either waist edge of the garment toward the crotch region. Each waistband 90 also exhibits a tension. The tension of waistbands employed in articles in accordance with the present invention is determined by the following test method, which is otherwise referred to herein as an elastic tension test or waistband tension test. The side seams of the product, if any, are first disengaged/cut open. Each product to be tested is hung from a conventional lightbox or other suitable device in an orientation such that the waistbands extend vertically, from floor to ceiling, with the bodyside liner of the absorbent assembly facing laterally outward away from the lightbox. The lightbox includes a pair of fixed upper clamps spaced apart to hold the front and back ends of the product. Each clamp should be approximately centered above each waistband. Each waistband should then be manually straightened and fully extended. Two lower clamp weights should then be attached to the bottom of the product centered beneath each waistband portion so that the product hangs in an extended configuration from the upper clamps. The spacing of the lower clamps should match the spacing of the upper clamps. The combined weight of the lower clamps and any hardware to which they are affixed is approximately 1,000 grams total mass. With the product in this extended configuration, a pair of markings is made on each waistband spaced along the length of the waistband from each other a distance of 100 mm, and the markings are symmetrical about the longitudinal centerline of the product. The clamp weight is then removed from the product and the product is removed from the lightbox. Each waistband is partially cut from the product by making two longitudinal cuts, each approximately 13 mm transversely outward of either pen mark. To complete removal of the waistband from the product, a transverse cut is made along the length of the waistband approximately 3 mm inward of the inner lengthwise edge of the waistband. Each test specimen (e.g., the marked and cut portion of each waistband) is then secured in a static tension testing device by a generally fixed upper clip and a generally moveable lower clip, both of which are constructed to inhibit the specimen against slipping or becoming damaged upon tensioning the specimen. More particularly, the specimen is first secured at one end by the upper clip, with the specimen marking near the one end being aligned with the leading edge (e.g., the lowermost edge) of the upper clip so that the specimen hangs freely from the upper clip in a generally relaxed (e.g., unstretched) condition. The weight of the specimen is tared and then the other end of the specimen is secured in the lower clip, with the marking near this other end of the specimen being aligned with the leading edge (e.g., uppermost edge) of the lower clip. The lower clip is then moved longitudinally away from the upper clip until the specimen is elongated longitudinally to about 90% of the previously achieved longitudinally elongated configuration (e.g., to a configuration in which the spacing between the markings on the specimen is about 90 mm, which is 90% of 100 mm) of the absorbent assembly. The specimen is maintained in this condition for approximately sixty seconds. The elastic tension is then measured and recorded using a suitable force gauge. The waistbands (i.e., both front and back) of at least five like products are tested using the identical procedure and the results are averaged to determine the elastic tension in the waistbands.

Such waistbands could be attached to be flush with the waist edge of the underlying chassis material, or could be “cantilevered” off the waist edge of the underlying chassis material, as is the case with the exemplary embodiments representatively illustrated in FIGS. 3 and 4. In the case of a waistband having two or more layers of material, the multiple layers can all be positioned on the bodyside of the chassis material, can all be positioned on the garment-side of the chassis material, or can be positioned in part on the bodyside of the chassis material and be positioned in part on the garment-side of the chassis material.

Desirably, in embodiments of the array of gender-specific absorbent articles employing waistbands, the waistbands are gender-specific in one or more respects. For example, the width (as defined above) of the waistbands in absorbent articles adapted for use by females can be different than the width of the waistbands in absorbent articles adapted for use by males. For example, to accommodate the style and fit preferences of men, the width 92 of the waistbands 90 in absorbent articles adapted for use by males is, in particular embodiments, greater than the width 92F of the waistbands 90F in absorbent articles adapted for use by females, as representatively illustrated in FIGS. 3 and 4. In another example, the tension (as defined above) of the waistbands in absorbent articles adapted for use by females can be different than the tension of the waistbands in absorbent articles adapted for use by males. For example, to accommodate the style and fit preferences of men, the tension of the waistbands in absorbent articles adapted for use by males is, in particular embodiments, greater than the tension of the waistbands in absorbent articles adapted for use by females.

In recognition of the fact that men and women have different style and tactile preferences, and frequently different skin and body hair amounts and characteristics, the gender-specific absorbent articles of the array of particular embodiments of the present invention can employ different materials. For example, the material used in the front portion and the back portion of the chassis of an article adapted for use by males can differ from the material used in the front and back portions of the chassis of an article adapted for use by females. For example, the chassis material in articles adapted for females may be more flexible, or have a smoother surface tactile quality, more akin to silk, than the chassis material in corresponding articles adapted for males. Other differences between chassis materials in products adapted for the different genders include substrate thickness, drapability, opacity, and basis weight.

Note too that many different components and subassemblies may be used in disposable, adult-incontinence articles. For example, rather than the hourglass-shaped liquid-impermeable and liquid-permeable substrates described above, a single, hourglass-shaped liquid-permeable substrate may be formed (e.g., two polypropylene facings attached to one another, with elastic strand disposed between the facings in certain locations, such as proximate to the leg openings, waist opening, and those portions of the front and back of the article that will contact the lower torso of a user). To this liquid-permeable substrate or chassis is attached an absorbent core or assembly, with the core or assembly comprising a cellulosic-fluff/superabsorbent material sandwiched between a liquid-impermeable backing, such as a film, and a liquid-permeable liner, such as a nonwoven. When the absorbent assembly is attached to the hourglass-shaped, liquid-permeable, chassis, the liquid-impermeable backing is attached to the chassis.

Other configurations are possible. For example, the article may be made by forming a front panel (or portion), a back panel (or portion), and attaching the two with a crotch portion comprising an absorbent assembly. And such articles may employ containment flaps, various fastening systems (including, e.g., refastenable systems such as those using hook-and-loop-type fasteners), separate waistbands attached to the article (as noted earlier), side panels (e.g., elastomeric side panels), and many other such components or assemblies. It should be readily understood that the present invention encompasses many such configurations of disposable articles, so long as said articles employ one or more of the identified inventive features which are varied between articles adapted to be worn by different genders (whether in the form of a product array or an individual article). Similarly, many different methods, unit operations, and processes may be used when making gender-specific, disposable, adult-incontinence articles. With respect to any of these features, the features may differ between the genders. For example, it may be desirable, for various product performance and/or manufacturing reasons, to employ a “three-piece” article design (such as the design described above comprising a front panel and a back panel indirectly attached together via an absorbent-assembly crotch portion) in articles adapted for use by one gender, but to employ a “one-piece” article design (such as the design described above in which a single, hourglass-shaped liquid-permeable substrate is provided, to which is attached an absorbent core or assembly) in articles adapted for use by the other, corresponding gender in the array. For example, to accommodate the style and fit preferences of females, it may be desirable in certain embodiments of an array of gender-specific absorbent articles to employ a “one-piece” chassis design in articles adapted for use by females, but to employ a “three-piece” chassis design in articles adapted for use by males.

As mentioned above, disposable absorbent articles of particular embodiments of the present invention may employ a pair of containment flaps that are configured to provide a barrier to the transverse flow of body exudates. A flap elastic member can be operatively joined with each containment flap in any suitable manner as is well known in the art. The elasticized containment flaps define an unattached edge which assumes an upright, generally perpendicular configuration to form a seal against the wearer's body. The opposing end of the flap is attached to the article (e.g., at a position inward of the leg-opening side edges). Suitable constructions and arrangements for the containment flaps are generally well known to those skilled in the art and are described, for example, in U.S. Pat. No. 4,704,116 issued Nov. 3, 1987 to Enloe. Accordingly, another representative version of the invention is an array of disposable absorbent articles comprising one or more of the dimensional (e.g., those inventive features directed to a length, width, distance, or other such measurement) or ornamental (e.g., those inventive features directed to disposing a graphic on an article) gender-specific features discussed above or below, with one or both gender-specific articles employing containment flaps.

With respect to the use of containment flaps, due to the differences in body function and insult location between males and females, it is desirable, in particular embodiments of the present invention, to employ structural differences in the containment flaps for products adapted for use by different genders. In particular embodiments, one gender-specific article has a single pair of containment flaps, while the other, corresponding gender-specific article in the array include two pairs of containment flaps. Such factors as the amount of absorbent material and the overall fit of the product may dictate which of the two products would have two pairs of containment flaps. In one example, absorbent articles adapted for use by males include two pairs of containment flaps, while absorbent articles adapted for use by females include one pair of containment flaps. In a contrasting example, absorbent articles adapted for use by females include two pairs of containment flaps, while absorbent articles adapted for use by males include one pair of containment flaps.

In another example, containment flaps employed in articles adapted for use by one gender may be folded transversely inward, while containment flaps employed in corresponding articles adapted for use by the other gender may be folded transversely outward. “Transversely inward” as used herein means in a direction toward the central longitudinal axis of the article. “Transversely outward” as used herein means in a direction away from the central longitudinal axis of the article. Such distinction between the genders can accommodate physical differences between the genders. For example, transversely folding the flaps outward may be more suitable for male users, in that the flaps may be expected to form a more “cup-like” shape, whereas transversely folding the flaps inward may be more suitable for female users, wherein such a design may be expected to cause less irritation in the female crotchal region.

In still another example, containment flaps employed in articles adapted for use by one gender may include a first number of layers of thickness, while containment flaps employed in corresponding articles adapted for use by the other gender may include a second number of layers of thickness. Such distinction between the genders can accommodate physical differences between the genders, and differences in the amount, force, and location of urine insults between the genders. For example, absorbent articles adapted for use by females may include containment flaps having a single layer of thickness, while absorbent articles adapted for use by males may include containment flaps having two, or even more, layers of thickness. In a contrasting example, absorbent articles adapted for use by males may include containment flaps having a single layer of thickness, while absorbent articles adapted for use by females may include containment flaps having two, or even more, layers of thickness.

It should be noted that when gender-specific constructions for various features are discussed herein, it is contemplated that, when a comparison is made between products having one or more design variables specifically tailored to males on the one hand and females on the other hand, the male and female products having those gender-specific features are designed for wearers of generally the same size. For example, if an adult incontinence garment is offered in three sizes for males (such as small, medium, and large), as well as three different sizes for females (such as small, medium and large), for a total of six different product codes, a comparison of any gender-specific design variable should be made for similarly sized products (e.g., compare male small to female small, male medium to female medium, and male large to female large). Likewise, if an adult incontinence garment is offered in varying absorbencies—such as low and high—each for both genders, a comparison of any gender-specific design variable should be examined for products of similar absorbency (e.g., compare male low absorbency to female low absorbency, and male high absorbency to female high absorbency) for a particular size.

Of course, the articles themselves are typically stored, shipped, and sold in packages, such as bags made of film. Certain representative versions of the array of disposable adult-incontinence articles of particular embodiments of the present invention can comprise one or more of the inventive dimensional (e.g., those inventive features directed to a length, width, distance, or other such measurement), ornamental (e.g., those inventive features directed to disposing a graphic on an article) or other gender-specific features discussed above, with the first disposable adult-incontinence article contained in a first package (e.g., a container such as a bag) having a first statement disposed thereon, wherein the first article is adapted to be worn by women; and a second disposable adult-incontinence article contained in a second package (e.g., a container such as a bag) having a second statement disposed thereon, wherein the second article is adapted to be worn by men; wherein the first statement associates the article with use by women and the second statement associates the article with use by men. The packages may also have different graphics, colors, symbols, logos, indicia, or other such representations, depending on whether the articles contained therein are adapted to be worn by men or women. Furthermore, for those gender-specific articles not part of an array, these same embodiments employing packages may be used for a single, gender-specific article.

For example, a package—which generally will be in the form of a container such as a plastic bag—containing disposable, adult-incontinence articles adapted to be worn by men may have: a picture of a man; text associating the article with use by a man; size and/or absorbency information specific to men; a graphic representation of the articles contained therein that, by virtue of the different graphics disposed on the articles themselves, are different—e.g., a graphic representation of an article adapted to be worn by men having one or more blue stripes proximate to the waist opening; text communicating to a user one or more features making the article gender-specific; or some combination thereof.

Similarly, in another exemplary version, a package containing disposable, adult-incontinence articles adapted to be worn by women may have: a picture of a woman; text associating the article with use by a woman; size and/or absorbency information specific to women; a graphic representation of the articles contained therein that, by virtue of the different graphics disposed on the articles themselves, are different—e.g., a graphic representation of an article adapted to be worn by women having one or more pink stripes proximate to the waist opening; text communicating to a user one or more features making the article gender-specific; or some combination thereof.

A manufacturer, seller, or distributor of the inventive array of gender-specific articles may seek to create awareness of said articles so that users enjoy the benefits of said gender-specific articles. Accordingly, another representative version of the invention is a method of providing a gender-specific, disposable absorbent article, the method comprising the steps of transmitting a statement embodied in a tangible medium that refers to the gender-specific, disposable absorbent article; and positioning in the hands of a user a disposable absorbent article adapted to fit the gender of the user.

A statement may be transmitted using a variety of communications, statements, messages, or copy, which could take the form of (i.e., be embodied in a medium such as) a newspaper advertisement, a television advertisement, a radio or other audio advertisement, items mailed directly to addressees, items emailed to addressees, Internet Web pages or other such postings, free standing inserts, coupons, various promotions (e.g., trade promotions), co-promotions with other companies, boxes or packages containing the product, and other such forms of disseminating information to consumers or potential consumers.

As noted above, some versions of the present invention may not contain an article adapted for use by males and an article adapted for use by females. Instead, some versions of the invention comprise a unisex, disposable absorbent article (such as an adult-incontinence article) and: (1) a disposable absorbent article (such as an adult-incontinence article) adapted to be worn by females comprising one or more of the inventive features described herein; or (2) a disposable absorbent article (such as an adult-incontinence article) adapted to be worn by males comprising one or more of the inventive features described herein. Various packaging and marketing techniques, including those described above, may be employed in conjunction with the aforementioned arrays comprising a unisex article.

Also, as noted above, the present invention encompasses gender-specific, disposable absorbent articles comprising one or more or a variety of gender-specific features. As with the aforementioned arrays, applicable packaging and marketing techniques, including those described above, may be employed in conjunction with these gender-specific articles. So, for example, in some versions of the invention, a disposable, adult-incontinence article adapted for women that comprises a gender-specific feature is contained in a container, and a statement disposed on said container associates the gender-specific feature with use of the article by women. In other versions of the invention, a disposable, adult-incontinence article adapted for men that comprises a gender-specific feature is contained in a container, and a statement disposed on said container associates the gender-specific feature with use of the article by men.

### EXAMPLES

The following example representatively illustrates a variety of features which could be used in conjunction with certain embodiments of the present invention. An array of gender-specific, disposable adult-incontinence articles was made. The articles were made using conventional materials and processes, and generally corresponded to the shapes depicted in FIGS. 1 and 2. More detailed drawings generally corresponding to those articles that were made are presented in FIGS. 3 (male article) and 4 (female article).

The materials employed included polypropylene nonwoven materials for much of the chassis construction; film as the moisture barrier/backsheet/backing for the absorbent assembly; an absorbent core comprising cellulosic fluff and superabsorbent material; and elastic strand around the waist opening, leg opening, and portions of the front and back panels/portions of the article. Hot-melt adhesive or ultrasonic energy was used to attach the components to one another. In this exemplary embodiment, a polypropylene nonwoven material having an hourglass-like shape served as the outer cover. To this were attached nonwoven front and back panels at the front and back portions of the article (with elastic strand sandwiched at various locations between these nonwoven front and back panels and the nonwoven outer cover). The front panel extended longitudinally from a location proximate to the waist-opening front edge to a location proximate to the crotch portion (but did not substantially overlay the crotch portion of the hourglass-like outer cover). The back panel extended longitudinally from a location proximate to the waist-opening back edge to a location proximate to the crotch portion (but, like the front panel, did not substantially overlay the crotch portion of the hourglass-like polypropylene outercover). Both the front panel and the back panel extended transversely from a location proximate to one side edge to the opposing side edge of the front portion and the back portion of the article.

An absorbent insert comprising a fluff/superabsorbent core sandwiched between a barrier/backsheet material (a film) and a body-side liner (a nonwoven material) was attached to the aforementioned assembly of an hourglass-like shaped outer cover having elastic strand sandwiched between front and back nonwoven panels at the front portion and the back portion of the article. Approximate dimensions and inter-relationships between the two articles are given in Table 2.

The anterior length 60 for the men's article was about 210 millimeters, and overlaid the side edge of the front portion, given that the leg-opening front edges were generally linear, and proceeded upward at a slight angle from the a location proximate to the crotch portion to the side edge itself (see FIG. 3). The anterior length 60F for the women's article was about 190 millimeters, however, and was measured from the point on the leg-opening front edge closest to the waist-opening front edge (see FIG. 4). This line, which is parallel to the longitudinal centerline of the article, is inward of the side edge of the front portion, given that the curvilinear boundary established by the leg-opening front edge of the women's article is more convex in a direction toward the waist opening front edge, compared to the corresponding, linear boundary established by the leg-opening front edge of the corresponding men's article. The leg-opening front edge of the women's article, having greater convexity, is better adapted to conform to the curved surface of the upper thigh region of a typical female user of such disposable, adult-incontinence articles.

An examination of FIG. 3 shows that the leg-opening back edges 62 of this exemplary version of an article adapted to be worn by men were also substantially linear, and proceeded downward at some angle from a location proximate to the crotch portion to the side edge of the back portion. The leg-opening back edge 62F of the exemplary women's article, however, does not define a substantially linear boundary, and instead defines a curvilinear boundary that is more concave in a direction toward the waist-opening back edge. The leg-opening back edge of the women's article, having greater concavity, is better adapted to ensure coverage of the buttocks region of a typical female user of such disposable, adult-incontinence articles.

Table 2 also shows that the perimeter of the leg-opening (which equates to the sum of the lengths of the leg-opening back, side, and front edges) for the women's article is greater than the perimeter of the leg-opening of the men's article. Again, this leg-opening feature is consistent with our observation that men, as they age, tend to lose muscle mass in their legs, resulting in many male adult-incontinence-product users typically having a smaller thigh circumference than many female adult-incontinence-product users.

Furthermore, Table 2 shows that the man's article has a crotch width and a gasket width greater than the crotch width and gasket width of the corresponding women's article. The gasket width 64 (see FIG. 3) of the men's article was about 40 millimeters and the gasket width of the women's article was about 20 millimeters (not shown on FIG. 4 due to the close proximity of the edge of the absorbent core to the outermost elastic member nearest the leg-opening side edge, but measured at the same location as the corresponding location on the men's article: along a line in a transverse direction intersecting the center of the leg-opening side edge).

Also, the location of the absorbent was shifted forward for the men's article, compared to the women's article, to accommodate the point at which urine typically exits a man's body compared to a women's body. Accordingly, the waist/absorbent distance 66 for the men's article was about 120 millimeters, and the waist/absorbent distance for the corresponding women's article 66F was about 180 millimeters. It should be noted that these distances were determined from the edge of the absorbent core (68 and 68F, respectively, in FIGS. 3 and 4). The edges 70 and 70F corresponds to the edge of the substrates encasing the absorbent core, with these substrates extending beyond the absorbent core itself to create a flexible flange surrounding the core. As discussed above, gasket width and waist/absorbent length/distance is measured from the edge of the absorbent core, not the edge of any substrate encasing and extending substantially beyond the edge of the absorbent core. It should also be noted that FIGS. 1 and 2 show the absorbent core, and a surge material, but not the outer perimeter of any substrate encasing the absorbent core and surge material. FIGS. 3 and 4, on the other hand, do not show a surge material, but do show both the absorbent core and the perimeter of the substrates used to encase the absorbent core.

Finally, while not shown, different colored stripes were disposed around each article at a location proximate to the waist opening. In this representative embodiment, a plurality of blue stripes was disposed around the perimeter of the men's article, and a plurality of pink stripes was disposed around the perimeter of the women's article.

TABLE 2

| Feature | Article for Women | Article for Men |
|---|---|---|
| Anterior Length | 190 mm | 210 mm |
| Shape of Leg-Opening Front | More convex toward | Less convex toward |
| Edge | waist-opening front | waist-opening front |
| | edge | edge |
| Shape of Leg-Opening Back | More concave | Less concave |
| Edge | toward waist- | toward waist- |
| | opening back edge | opening back edge |
| Leg-Opening Perimeter | 785 mm | 740 mm |
| Crotch Width | 155 mm | 195 mm |
| Waist/Absorbent Distance | 180 mm | 120 mm |
| Frontal Area of Absorbent | Less | More |
| Core | | |
| Graphic Disposed on Article | Pink-colored stripes | Blue-colored stripes |
| | proximate to waist | proximate to waist |
| | opening | opening |
| Gasket Width | 20 mm | 40 mm |

We claim:

1. An array of gender-specific, disposable, absorbent articles, the array comprising:
a package of first disposable absorbent articles, the package bearing indicia indicating that the articles therein are adapted to be worn by females, each first disposable absorbent article having a first product length;
a package of second disposable absorbent articles, the package bearing indicia indicating that the articles therein are adapted to be worn by males, each second disposable absorbent article having a second product length;
wherein the first product length is different from the second product length.

2. The array of claim 1, wherein the first product length is less than the second product length.

3. The array of claim 1, wherein the first product length is greater than the second product length.

4. An array of gender-specific, disposable, absorbent articles, the array comprising:
a package of first disposable absorbent articles, the package bearing indicia indicating that the articles therein are adapted to be worn by females, each first disposable absorbent article having a first crotch length;
a package of second disposable absorbent articles, the package bearing indicia indicating that the articles therein are adapted to be worn by males, each second disposable absorbent article having a second crotch length;
wherein the first crotch length is different from the second crotch length.

5. The array of claim 4, wherein the first crotch length is less than the second crotch length.

6. The array of claim 4, wherein the first crotch length is greater than the second crotch length.

7. An array of gender-specific, disposable, absorbent articles, the array comprising:
a package of first disposable absorbent articles, the package bearing indicia indicating that the articles therein are adapted to be worn by females;
a package of second disposable absorbent articles, the package bearing indicia indicating that the articles therein are adapted to be worn by males;
wherein either each first disposable absorbent article has a first graphic visible when viewing an inner, body-facing surface of the first disposable absorbent article, each second disposable absorbent article has a second graphic visible when viewing an inner, body-facing surface of the second disposable absorbent article, or both each first disposable absorbent article has a first graphic visible when viewing the inner, body-facing surface of the first disposable absorbent article and each second disposable absorbent article has a second graphic visible when viewing the inner, body-facing surface of the second disposable absorbent article.

8. The array of claim 7, wherein each first disposable absorbent article has a graphic visible when viewing its inner, body-facing surface, and each second disposable absorbent article does not have a graphic visible when viewing its inner, body-facing surface.

9. The array of claim 7, wherein each second disposable absorbent article has a graphic visible when viewing its inner, body-facing surface, and each first disposable absorbent article does not have a graphic visible when viewing its inner, body-facing surface.

10. The array of claim 7, wherein each first disposable absorbent article has a first graphic visible when viewing its inner, body-facing surface, and each second disposable absorbent article has a second graphic visible when viewing its inner, body-facing surface, wherein the first graphic and the second graphic are substantially different.

11. An array of gender-specific, disposable, absorbent articles, the array comprising:
a package of first disposable absorbent articles, the package bearing indicia indicating that the articles therein are adapted to be worn by females;
a package of second disposable absorbent articles, the package bearing indicia indicating that the articles therein are adapted to be worn by males;
wherein the package of first disposable absorbent articles has a first fragrance, and the package of second disposable absorbent articles has either (1) a second fragrance different from the first fragrance or (2) no fragrance.

12. The array of claim 11, wherein the package of second disposable absorbent articles has a second fragrance different from the first fragrance.

13. The array of claim 11, wherein the package of second disposable absorbent articles has substantially no fragrance.

14. An array of gender-specific, disposable, absorbent articles, the array comprising:
a package of first disposable absorbent articles, the package bearing indicia indicating that the articles therein are adapted to be worn by females, each first disposable absorbent article having a first leg elastic member;
a package of second disposable absorbent articles, the package bearing indicia indicating that the articles therein are adapted to be worn by males, each second disposable absorbent article having a second leg elastic member;
wherein the first leg elastic member extends along a first path and the second leg elastic member extends along a second path that has a different shape than the first path.

15. The array of claim 14, wherein the first path is curved, and the second path are straight.

16. The array of claim 14, wherein the first path is straight, and the second path is curved.

17. An array of gender-specific, disposable, absorbent articles, the array comprising:
a package of first disposable absorbent articles, the package bearing indicia indicating that the articles therein are adapted to be worn by females, each first disposable absorbent article having a separately attached first waistband;
a package of second disposable absorbent articles, the package bearing indicia indicating that the articles therein are adapted to be worn by males, each second disposable absorbent article having a separately attached second waistband;
wherein the first waistband differs from the second waistband in at least one of width and tension.

18. The array of claim 17, wherein a first waistband width is different than a second waistband width.

19. The array of claim 18, wherein the first waistband width is less than the second waistband width.

20. The array of claim 17, wherein a first waistband tension is different than a second waistband tension.

21. The array of claim 20, wherein the first waistband tension is less than the second waistband tension.

22. An array of gender-specific, disposable, absorbent articles, the array comprising:
a package of first disposable absorbent articles, the package bearing indicia indicating that the articles therein are adapted to be worn by females, each first disposable absorbent article having a first chassis design;
a package of second disposable absorbent articles, the package bearing indicia indicating that the articles therein are adapted to be worn by males, each second disposable absorbent article having a second chassis design;
wherein the first chassis design is different than the second chassis design.

23. The array of claim 22, wherein each first disposable absorbent article comprises a front portion, a back portion, and a crotch portion, wherein the front portion, the back portion, and the crotch portion are integrally connected in the first chassis design, and wherein each second disposable absorbent article comprises a front portion, a back portion, and a crotch portion, wherein the front portion is indirectly and non-integrally connected to the back portion by way of the crotch portion positioned longitudinally intermediate to and bonded to both the front portion and the back portion in the second chassis design.

24. An array of gender-specific, disposable, absorbent articles, the array comprising:
a package of first disposable absorbent articles, the package bearing indicia indicating that the articles therein are adapted to be worn by females, each first disposable absorbent article having a set of first containment flaps;
a package of second disposable absorbent articles, the package bearing indicia indicating that the articles therein are adapted to be worn by males, each second disposable absorbent article having a set of second containment flaps;
wherein the set of first containment flaps differs in at least one structural aspect from the set of second containment flaps.

25. The array of claim 24, wherein the set of first containment flaps consists of one pair of containment flaps, and wherein the set of second containment flaps consist of two pairs of containments flaps.

26. The array of claim 24, wherein each of the first containment flaps is fold transversely inward, and wherein each of the second containment flaps is folded transversely outward.

27. The array of claim 24, wherein each of the first containment flaps includes only one layer of material thickness, and wherein each of the second containment flaps includes at least two layers of material thickness.
